(19) United States
(12) Patent Application Publication
(10) Pub. No.: US 2020/0282146 A1
(43) Pub. Date: Sep. 10, 2020

(54) SINGLE-USE SYRINGE

(71) Applicant: Neo Mechanics Limited, Hong Kong (HK)

(72) Inventors: Stanley Moon Kai Ko, Hong Kong (HK); Kyong Tae Chang, Seoul (KR)

(21) Appl. No.: 16/297,650

(22) Filed: Mar. 9, 2019

(51) Int. Cl. A61M 5/315; A61M 5/31

(52) U.S. Cl. CPC A61M 5/31515; A61M 2210/1003; A61M 5/3135; A61M 5/31513

(57) ABSTRACT

A single-use syringe, comprising a barrel, and a first resilient unidirectionally-biased peripheral annulus formed inside the barrel; an upper plunger, having a dome shaped bottom mounted at a first end of the upper plunger, a first flange formed at a second end of the upper plunger with a second flange formed adjacent to it; a lower plunger having a second resilient unidirectionally-biased peripheral annulus formed near a top end of the lower plunger and a third resilient unidirectionally-biased peripheral annulus formed further inside the lower plunger, wherein a holding space is defined by a space between the second annulus and the third annulus, allowing the dome shaped bottom be inserted through the second annulus, held in the holding space, and pressed against the third annulus during a syringe unlocked state, and wherein a locking chamber is defined by a space between the third annulus and the lower plunger bottom end.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent Office patent file or records, but otherwise reserves all copyright rights whatsoever.

### FIELD OF THE INVENTION

The present invention relates to disposable and single-use syringes.

### BACKGROUND OF THE INVENTION

The need for a single-use syringe comes from the concern in the transfer of infectious diseases such as AIDS, and more particularly in some intravenous drug users who routinely share and reuse syringes. Although there are certain regulations and procedures for the proper disposal of used syringes, failure of following such regulations and procedures often occur, many due to emergency circumstances, negligence, and abuse.

There are many different designs of single-use syringes in the art. However, none possesses all the desirable features of user friendliness, simplicity, and reliability in both usage and fabrication. Lacking self-containments of all parts is also an impediment to the wide adoption of single-use syringes.

### SUMMARY

It is the objective of the present invention to provide a single-use syringe that addresses the aforementioned and other needs. Embodiments of the present invention can be used as epidural needles and other applications of fluid and gas discharging.

In accordance to an embodiment of the present invention, provided is a syringe comprises a barrel resembling a cylindrical tube, an upper plunger, a lower plunger, a plunger seal, and a needle adaptor or nozzle. The barrel comprises an open top end, a bottom end connected to the needle adaptor or nozzle, and a first resilient unidirectionally-biased peripheral annulus formed inside the barrel and approximately near the top end opening.

The upper plunger is configured for moving axially inside the barrel. The upper plunger has a dome or cone shaped bottom mounted at a first end of the upper plunger, a first flange formed at a second end and a second flange formed adjacent to the first flange.

The lower plunger is a cylindrical tube with an opened first end and an opened or closed second end. The lower plunger comprises a second resilient unidirectionally-biased peripheral annulus formed near the first end and a third resilient unidirectionally-biased peripheral annulus formed further inside the lower plunger. The space between the second annulus and the third annulus defines a holding chamber that holds the dome or cone shaped bottom of the upper plunger. The plunger seal is installed at the second end of the lower plunger and is intimate contact with the inner wall of the barrel, providing the sealing function.

Before the fluid or gas intake and expel actions, the upper plunger is connected with the lower plunger in that the dome or cone shaped bottom of the upper plunger is inserted into the lower plunger, passing the second resilient unidirectionally-biased peripheral annulus and just pressing against the third resilient unidirectionally-biased peripheral annulus. The third resilient unidirectionally-biased peripheral annulus provides just enough resilience against the pushing force of the dome or cone shaped bottom of the upper plunger to actuate the lower plunger when the upper plunger is inserted further into the barrel during fluid or gas expel action.

When the lower plunger reaches and stops at the bottom end of the barrel, further pushing force of the dome or cone shaped bottom of the upper plunger against the third resilient unidirectionally-biased peripheral annulus causes the dome or cone shaped bottom to breach the third annulus, and simultaneously the second flange to breach first resilient unidirectionally-biased peripheral annulus inside the barrel. The dome or cone shaped bottom is then entirely trapped inside the lower plunger, and the second flange is trapped by the first resilient unidirectionally-biased peripheral annulus inside the barrel. As such, the upper plunger is locked in the barrel, preventing any subsequent fluid/gas intake action.

### BRIEF DESCRIPTION OF THE DRAWINGS

The present invention is illustrated by way of example, and not by way of limitation, in the figures of the accompanying drawings in which like reference numerals refer to similar elements and in which:

FIG. 1 is an exploded view of a syringe in accordance with an embodiment of the present invention;

FIG. 2 is a close-up perspective view of a barrel of the syringe in accordance with an embodiment of the present invention;

FIG. 3 is a close-up perspective view of a lower plunger of the syringe in accordance with an embodiment of the present invention; and

FIGS. 4, 5, 6, and 7 are schematic diagrams illustrating the usage of the syringe in accordance with an embodiment of the present invention.

### DETAILED DESCRIPTION

In the following description, syringes and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

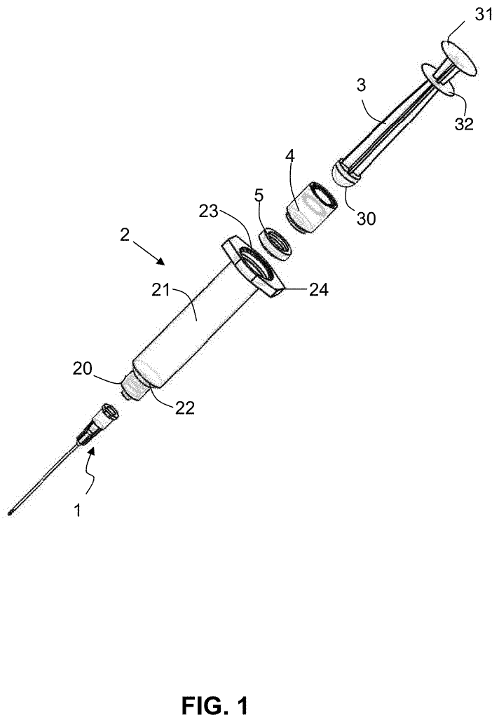

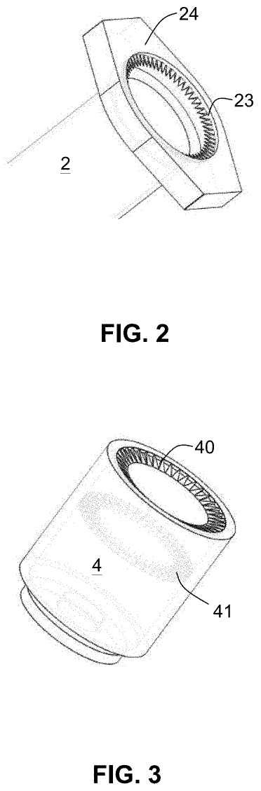

Referring to FIGS. 1 and 2. FIG. 1 is an exploded view of a syringe in accordance with an embodiment of the present invention; FIG. 2 is a close-up view of the barrel of the syringe in accordance with an embodiment of the present invention. As shown in FIGS. 1 and 2, a syringe 2 is provided, which comprises a barrel 21 resembling a cylindrical tube, an upper plunger 3, a lower plunger 4 and a plunger seal 5, and a needle adaptor or nozzle 20. If the syringe 2 is configured for the use of an epidural needle, a needle assembly 1 is connected to the needle adaptor 20.

The barrel 21 comprises an open top end, a bottom end having a shoulder 22 and connected to the needle adaptor or nozzle 20, and a first resilient unidirectionally-biased peripheral annulus 23 formed inside the barrel 21 and approximately near the top end opening.

The upper plunger 3 is configured for moving axially inside the barrel 2. The upper plunger 3 has a dome or cone shaped bottom 30 mounted at a first end of the upper plunger 3, a first flange 31 formed at a second end and a second flange 32 formed adjacent to the first flange 31.

As shown in FIG. 2, in one embodiment, the first resilient unidirectionally-biased peripheral annulus 23 of the barrel 21 comprises multiple transverse protuberances, and each protuberance projecting downwardly from an inner wall of the barrel 21. The third flange 24 is formed on an outer wall of the barrel 21 corresponded to the first resilient unidirectionally-biased peripheral annulus 23.

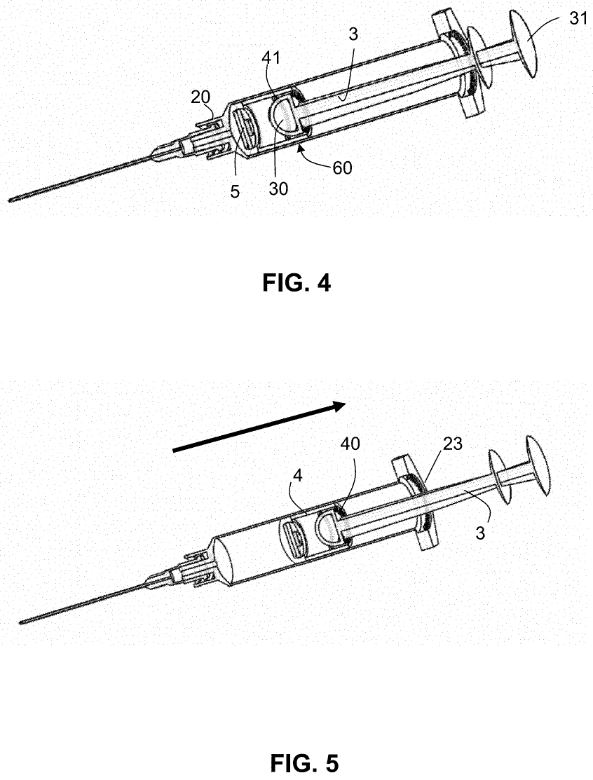

With further reference to FIG. 3. FIG. 3 is a close-up perspective view of a lower plunger of the syringe in accordance with an embodiment of the present invention. In this embodiment, the lower plunger 4 is a cylindrical tube with an opened first end and an opened or closed second end. The lower plunger 4 comprises a second resilient unidirectionally-biased peripheral annulus 40 formed near the first end and a third resilient unidirectionally-biased peripheral annulus 41 formed further inside the lower plunger 4. The space between the second resilient unidirectionally-biased peripheral annulus 40 and the third resilient unidirectionally-biased peripheral annulus 41 defines a holding chamber that holds the dome or cone shaped bottom 30 of the upper plunger 3. The plunger seal 5 installed at the second end of the lower plunger 4 and is intimate contact with the inner wall of the barrel 21, providing the sealing function.

Before the fluid or gas intake and expel actions, the upper plunger 3 is connected with the lower plunger 4 in that the dome or cone shaped bottom 30 of the upper plunger 3 is inserted into the lower plunger 4, passing the second resilient unidirectionally-biased peripheral annulus 40 and just pressing against the third resilient unidirectionally-biased peripheral annulus 41. The third resilient unidirectionally-biased peripheral annulus 41 provides just enough resilience against the pushing force of the dome or cone shaped bottom 30 of the upper plunger 3 to actuate the lower plunger 4 when the upper plunger 3 is inserted further into the barrel during fluid or gas expel action.

When the lower plunger 4 reaches and stops at the bottom end of the barrel 21, further pushing force of the dome or cone shaped bottom 30 of the upper plunger 3 against the third resilient unidirectionally-biased peripheral annulus 41 causes the dome or cone shaped bottom 30 to breach the third annulus 41, and simultaneously the second flange 32 to breach first resilient unidirectionally-biased peripheral annulus 23 inside the barrel 21. The dome or cone shaped bottom 30 is then entirely trapped inside the lower plunger 4 passed the third annulus 41 (in the locking chamber, which is the space between the third annulus 41 and the second end of the lower plunger 4), and the second flange 32 is trapped by the first annulus 23 inside the barrel. As such, the upper plunger 3 is locked in the barrel 21, preventing any subsequent fluid/gas intake action.

In accordance to one embodiment, the second resilient unidirectionally-biased peripheral annulus 40 is formed with multiple transverse protuberances, and each protuberance projecting downwardly from an inner wall of the lower plunger 4. The third resilient unidirectionally-biased peripheral annulus 41 comprises multiple transverse protuberances projecting radially from the inner wall of the lower plunger.

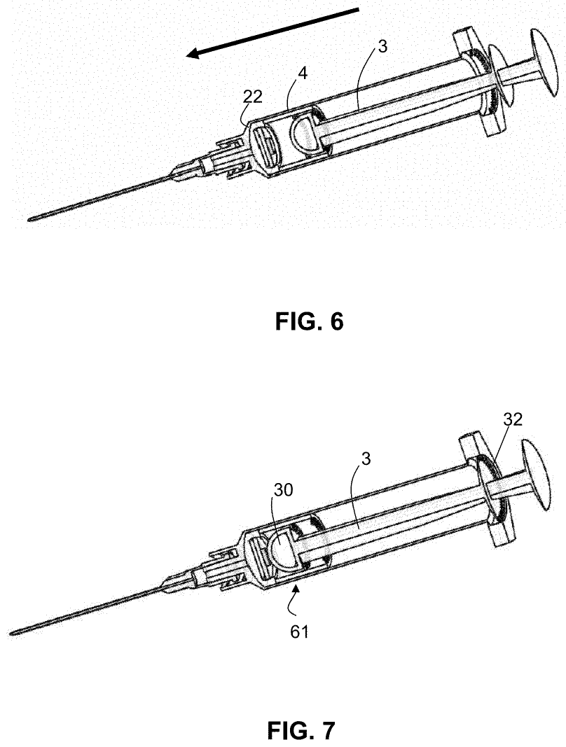

Referring to FIGS. 4 to 7. FIG. 4 illustrates the syringe in an initial state in accordance with an embodiment of the present invention; FIG. 5 illustrates the syringe is in a pulling state; FIG. 6 illustrates the syringe is in a pushing state; and FIG. 7 illustrates the syringe in a locked state.

Initially, as shown in FIG. 4, the assembly of the upper plunger 3, lower plunger 4, and the plunger seal 5 is moved toward the needle adaptor or nozzle 20 that connects to the bottom end of the barrel 21 (e.g., the shoulder 22 of the barrel 21), which in order to remove all air before a fluid/gas intake action. The dome or cone shaped bottom 30 is firmly held inside the holding portion 60. In other words, the lower plunger 4 is engaged to the upper plunger 3.

The third resilient unidirectionally-biased peripheral annulus 41 of the lower plunger 4 prevents the upper plunger 3 going totally inside the lower plunger 4 when a person pushing too hard on the first flange 31 for removing air.

Filling the syringe with medicine after air being discharged, as shown in FIG. 5, the upper plunger 3 is pulled with the lower plunger 4 moving toward the first resilient unidirectionally-biased peripheral annulus 23. The upper plunger 3 stopped when the fluid or gas is drawn up to a desired amount. The first resilient unidirectionally-biased peripheral annulus 23 ensures the upper plunger 3 cannot be pulled out from the barrel 21, and the second resilient unidirectionally-biased peripheral annulus 40 prevents the upper plunger 3 being pull out from the lower plunger 4.

In a fluid or gas expel action, the upper plunger 3 is then pushed, as shown in FIG. 6, to actuate the lower plunger 4 to move toward the shoulder 22 until the plunger seal 5 reaches the bottom end of the barrel 21 to complete the entire fluid or gas expel.

After the completion of the fluid or gas expel, as shown in FIG. 7, the upper plunger 3 is then pushed further to force the dome or cone shaped bottom 30 pass the third resilient unidirectionally-biased peripheral annulus 41. The dome or cone shaped bottom 30 is then entirely trapped inside the locking portion 61 of the lower plunger 4. Simultaneously, the second flange 32 breaches the first resilient unidirectionally-biased peripheral annulus 23 inside the barrel. Eventually, the upper plunger 3 is locked, preventing the upper plunger 3 from pulling out of the barrel 21. As such, the syringe is rendered usable.

It is noted that the first, second, and third resilient unidirectionally-biased peripheral annulus are formed with multiple transverse protuberances projecting downwardly from the surrounding walls. Once the upper plunger is locked, applying external force for detachment will break the protuberances. A skill person in the art can appreciate that other mechanical mechanisms may be adopted in place of the multiple transverse protuberances to achieve the same function without undue experimentation or deviation from the spirit of the present invention.

The embodiments described above do not require any additional and/or external means or components to prevent the syringe from being reused once locked. Thus, the syringe of the present invention provides the desirable features of user friendliness, simplicity, and reliability in both usage and fabrication, along with a self-containment of all parts.

While the disclosure has been described in connection with a number of embodiments and implementations, the disclosure is not so limited but covers various obvious modifications and equivalent arrangements, which fall within the purview of the appended claims. Although features of the disclosure are expressed in certain combinations among the claims, it is contemplated that these features can be arranged in any combination and order.

What is claimed is:

1. A syringe, comprising:
a barrel having an open top end, a bottom end connected to a needle adaptor or nozzle, and a first resilient unidirectionally-biased peripheral annulus formed inside the barrel;
an upper plunger movable in the barrel, having a dome or cone shaped bottom mounted at a first end of the upper plunger, a first flange formed at a second end of the upper plunger and a second flange formed adjacent to the first flange;
a lower plunger having a second resilient unidirectionally-biased peripheral annulus formed near a top end of the lower plunger and a third resilient unidirectionally-biased peripheral annulus formed further inside the lower plunger,
wherein a holding space is defined by a space between the second resilient unidirectionally-biased peripheral annulus and the third resilient unidirectionally-biased peripheral annulus, allowing the dome or cone shaped bottom be inserted through the second resilient unidirectionally-biased peripheral annulus, be held in the holding space, and be pressed against the third resilient unidirectionally-biased peripheral annulus during a syringe unlocked state, and
wherein a locking chamber is defined by a space between the third resilient unidirectionally-biased peripheral annulus and a bottom end of the lower plunger; and
a plunger seal connected to the lower plunger.

2. The syringe as claimed in claim 1, wherein during the syringe locked stage, the upper plunger is locked when the upper plunger is pushed further advanced toward the bottom end of the barren to cause the second flange to breach the first resilient unidirectionally-biased peripheral annulus inside the barrel, and the dome or cone shaped bottom to move into the locking chamber.

3. The syringe as claimed in claim 1, wherein the first resilient unidirectionally-biased peripheral annulus comprises multiple transverse protuberances, each protuberance projecting downwardly from an inner wall of the barrel.

4. The syringe as claimed in claim 1, wherein the barrel further comprises a third flange formed on an outer wall of the barrel corresponded to the first resilient unidirectionally-biased peripheral annulus.

5. The syringe as claimed in claim 1, wherein the second resilient unidirectionally-biased peripheral annulus comprises multiple transverse protuberances, each protuberance projecting downwardly from an inner wall of the lower plunger.

6. The syringe as claimed in claim 1, wherein the third resilient unidirectionally-biased peripheral annulus comprises multiple transverse protuberances projecting radially from the inner wall of the lower plunger.
